(19) United States
(12) Patent Application Publication
(10) Pub. No.: US 2024/0237972 A1
(43) Pub. Date: Jul. 18, 2024

(54) Self-Diagnostic Ovulation Tool

(71) Applicant: Khyatee Tewari, Alexandria, VA (US)

(72) Inventor: Khyatee Tewari, Alexandria, VA (US)

(21) Appl. No.: 18/539,986

(22) Filed: Dec. 14, 2023

(51) Int. Cl. A61B 10/00

(52) U.S. Cl. CPC A61B 10/0012; A61B 2010/0006; A61B 2010/0032

(57) ABSTRACT

A user-friendly, non-invasive method for swift screening of vaginal discharge to identify the fertility window by placing or attaching a pH sensitive strip or solution on the crotch panel of the user's underwear. The purpose of the pH sensitive strip is to allow the user to monitor her ovulation cycle in order to inform household decisions related to prevention or promotion of pregnancy.

### TECHNICAL FIELD

The invention is a non-invasive method that can be used to detect the fertility window of a woman, allowing her to make informed decisions about family planning.

### SUMMARY

The disclosed invention functions as an end-user focused disposable method to indicate fertility window and other health conditions in women. The primary objective of this invention is to be able to provide a novel method that can be used by women, to understand their ovulation cycle, and, in extension, their reproductive health.

It is a non-invasive, one-time ready to use disposable strip or solution, which would be placed, by the user, on the crotch panel of their undergarment. It consists of a pH sensitive solution that can indicate a woman's (the intended end-user) fertility window to enable an active choice in promotion and prevention of pregnancies.

As a woman progresses through the menstruation cycle, frequently exuding small amounts of vaginal discharge, and enters the ovulation phase, the pH of the aforementioned vaginal discharge turns neutral-basic (pH of >7) from acidic (pH<7).

The intention of the invention is to enable the user to attach the pH sensitive disposable strip or solution on the crotch panel of her underwear and receive feedback through the change, or lack thereof, in the color of the strip or solution. A change in the color will inform the user that the pH value of the vaginal discharge has increased (i.e. vaginal discharge has neutralized) thereby, indicating that the user has entered the ovulation phase. Following this, users will be probed for data about the change in color which would, then, be used to provide advice and feedback at a macro and micro level respectively.

### BACKGROUND

Use of different contraception methods to assist in family planning have been widely explored and distributed in the past few decades. Despite the increase in adoption of family planning methods across various countries, there is an underinvestment in women's health thereby disempowering women with regard to their decision-making (pertaining to reproductive health).

Contraceptive methods like condoms are male-dependent, restricting the woman's control over her reproductive cycle. Methods such as IUDs and birth control pills are intrusive to women's bodies, often making their usage stigmatized and uncomfortable. Other modern methods such as mobile applications with period trackers and menstruation cycle predictors are urban centered, limiting their usage to women owning smartphones. These solutions have not focused on the lack of awareness, unaffordability, and social stigmas that are associated with these modern family planning methods. Additionally, most of the contraception methods require a positive action to be undertaken by the woman to either control or alter her hormones, frequently accompanied by many common and harmful side-effects.

This invention draws its inspiration from Hydrangea flowers which change color depending on the potential of Hydrogen, or the pH value of the soil they are planted in. Similar to the flower, the invention, when placed in the crotch panel of the user's underwear, will change color to indicate the change in pH of the vaginal discharge of the user. The pH of the vaginal discharge generally fluctuates between 3.8 to 4.5, being moderately acidic for most of the cycle. The acidic nature of the discharge protects it from bacteria and other infections that grow best at a pH of about 7.5. However, during ovulation, there is a luteinizing hormone surge which increases the pH level of the discharge to above 7 (alkaline in nature), in order to provide a safe environment for the sperm to enter and fertilize the egg. This property of the vaginal discharge is relatively underexplored and has not been considered for creation of methods to promote family planning.

This invention, however, uses the property of LH surges in the vaginal discharge to indicate time of ovulation through a pH sensitive fabric/paper or solution which may be worn throughout the day (by attaching it on the crotch panel of the underwear) and does not require, for instance, conscious effort to test during urination. It also appreciates the constraints of affordability, social stigma, and financial and geographical dependencies that restrict women's abilities to access reproductive and family planning methods.

### DETAILED DESCRIPTION

Concept Validation: Nakano et al in their study on the ‘Insights into the role of cervical mucus and vaginal pH in unexplained infertility’ explain the importance of the pH level of the vaginal discharge for fertility. The study examines the varying levels of the pH during a woman's menstrual cycle concluding that the vaginal discharge is acidic for most part of the cycle, except during ovulation, when it becomes alkaline, in order to facilitate fertilization. This was observed in our preliminary experiment phase where we compared different pH levels of the vaginal discharge for a month, in three women, and found that pH level of the discharge increased to greater than 7 during ovulation.

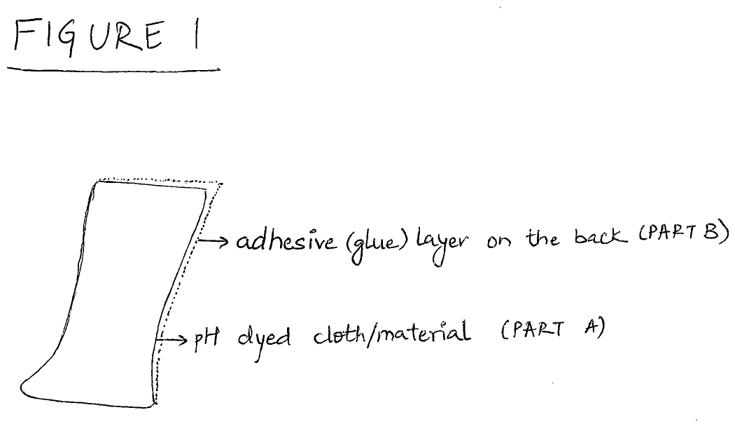

Invention description: FIG. 1 represents the invention consisting of two parts; Part A and Part B. Part A is a common use piece of fabric or paper that is impregnated with a pH sensitive solution which changes color when the pH value of the vaginal discharge/fluid increases beyond 6. The shape of the fabric/paper will mimic the shape of a generic crotch panel on the underwear and be limited to its size. The change in pH value beyond 6 induces a change in the color of the fabric/paper which can be juxtaposed with the original color and the color chart that will be provided along with the invention; either in the form of a booklet or directly on the packaging of the invention. This color change will be visible to the user when the vaginal discharge from a woman's body comes in contact with the strip.

Part B represents the back of the pH sensitive strip which will be layered with adhesive glue and covered with detachable paper, which when removed exposes the adhesive that can be used to stick/attach the strip onto the crotch panel of the underwear. After use, the strip can be easily removed, disposed, and replaced.

Wearing: The fabric/paper strip or solution can be placed on the crotch panel of the underwear by removing the paper cover and placing onto the panel in a way that fits the shape so as to receive the vaginal discharge. Part A of the invention must be placed facing upwards, whereas Part B should be used to stick to the fabric of the undergarment. The fabric/paper can be worn for the whole day, unless the discharge is of a greater volume and/or occurs more frequently than average, at which point, the fabric/paper can be replaced with another one.

Observing and comparing: Once the fabric/paper or solution has been placed on the crotch panel, the user can observe the change in color (if any) by comparing to the area which has not been touched with secretions or with the color chart provided along with the invention; either on the product cover or as a separate booklet. The change in the color of the fabric/paper indicates the pH level of the discharge i.e. if it is acidic or basic. When the fabric/paper indicates that the discharge is basic, it can be assumed that the user has entered her ovulation phase (i.e. the fertility window has opened). Peak ovulation period lasts from about 12 hours to 48 hours, however, women remain fertile for about seven days. The invention has the ability to be able to indicate the fertility window before and beyond the peak days, until the discharge returns to its otherwise acidic nature.

Removing and disposing: The user can remove the fabric/paper at the same time as they would otherwise change their undergarment. This can be done by removing the fabric/paper from the crotch panel, the same way as they would remove a sanitary pad or panty liner. This fabric/paper can then be disposed of and a new strip can be placed to continue measuring the pH level.

Data collection: Following primary distribution and implementation of the invention, the next phase will involve data collection, and feedback to the users. The methods for data collection are split into two types, targeting two identified segments of women.

The first method targets urban, higher income women, who have access to smartphones and moderate connectivity to the internet. This method will prompt the user, via a mobile application, to enter the color of the disposable strip after every use of the strip. A color wheel will be included within the application to allow the user to easily identify and select the color on or after the day of use.

The second, and complementary, method, targets lower income women, specifically in rural areas who do not have access to either smartphones or reliable internet connectivity. This method will require a prompt action from the user, based on the corresponding color displayed on the packaging of the invention, after each daily use of the strip. The codes of the color will correspond to the same color wheel as provided to the former segment of users to maintain consistency in data collection and allow for efficient and helpful aggregation and feedback.

### CONCLUSION

This invention aims to provide an easy and convenient way for women to take control of their reproductive health and make informed decisions about family planning. The invention, unlike other contraceptive methods, is non-obtrusive, has no side effects, and is less expensive than other alternatives. The overarching aim of the invention is to provide a method to collect data and provide information regarding reproductive health.

What is claimed is as follows:

1. A one-time ready to use method, to indicate the position in menstruation cycle using pH characteristic due to LH surges in the vaginal discharge, consisting of:—
1. An organic pH sensitive solution that can be used on a fabric/paper or on the underwear crotch panel itself to make it pH sensitive.
2. A self-diagnostic method of identifying the fertility window throughout the ovulation cycle.
a. Wearing a pH sensitive fabric/paper shaped like the crotch panel of the underwear to receive vaginal discharge
b. Observing the color change indicated by increase in pH value of vaginal discharge
c. Comparing the color change to determine if one is ovulating or not
d. Removing and disposing the strip after vaginal discharge has been received and color has been observed
3. A self-diagnostic product which uses the following combinations to check the change in pH level due to LH surges in the vaginal discharge indicating the fertility window.
a. A fabric/paper that consists of pH sensitive solution on one side, which is sensitive to different pH levels
b. An adhesive glue on the other side to facilitate positioning of fabric/paper on the underwear
4. A self-diagnostic product for which results are easy to report by the end user.
a. A smart phone application with a color wheel to report the change in color of the product
b. A self-report method using mobile phone network to report the change in color based on codes provided on the packaging
